(19) United States
(12) Patent Application Publication
(10) Pub. No.: US 2025/0143723 A1
(43) Pub. Date: May 8, 2025

(54) ASPIRATION CATHETER

(71) Applicant: Boston Scientific Scimed, Inc., Maple Grove, MN (US)

(72) Inventors: Anthony Frank Tassoni, JR., Andover, MN (US); Robert D. Cooper, Andover, MN (US); Alyssa Madej, Minnetonka, MN (US); Eric Samuel Rowley, Otsego, MN (US); David Brajkovic, Minneapolis, MN (US)

(73) Assignee: Boston Scientific Scimed, Inc., Maple Grove, MN (US)

(21) Appl. No.: 18/933,089

(22) Filed: Oct. 31, 2024

Related U.S. Application Data
(60) Provisional application No. 63/547,205, filed on Nov. 3, 2023.

(51) Int. Cl. A61B 17/22; A61M 25/00; A61M 25/09

(52) U.S. Cl. CPC A61B 17/22; A61M 25/0009; A61M 25/0023; A61M 25/09; A61B 2017/22079

(57) ABSTRACT

This disclosure provides design, material, manufacturing method, and use alternatives for medical devices. An example aspiration catheter includes a catheter shaft having a proximal end region, a distal end region and a lumen extending therein. The aspiration catheter also includes a tip member having a length and a lumen extending therein, wherein the tip member is coupled to the distal end region of the catheter shaft and a guidewire shaft having a proximal end region and a distal end region. Further, the guidewire shaft extends within at least a portion of the lumen of the catheter shaft and the lumen of the tip member. Further, the distal end region of the guidewire shaft includes an interlock region and a portion of the tip member is engaged with at least a portion of the interlock region.

### CROSS-REFERENCE TO RELATED APPLICATIONS

This application claims the benefit of priority under 35 U.S.C. § 119 of U.S. Provisional Application No. 63/547,205, filed Nov. 3, 2023, the entire disclosure of which is hereby incorporated by reference.

### TECHNICAL FIELD

The disclosure is directed to aspiration systems. More particularly, the disclosure is directed to an aspiration catheter having a strengthened distal tip bond.

### BACKGROUND

Thrombectomy is a procedure for removing thrombus from the vasculature of a patient. Mechanical and fluid-based systems can be used to remove thrombus. With fluid-based systems, an infusion fluid may be infused to a treatment area of a vessel with a catheter to dislodge the thrombus. In some instances, an effluent (e.g., the infusion fluid and/or blood) including the dislodged thrombus may be extracted from the vessel through the catheter. Of the known thrombectomy systems and methods, there is an ongoing need to provide alternative configurations of thrombectomy catheters and systems, as well as methods of operating such thrombectomy systems.

### SUMMARY

This disclosure provides design, material, manufacturing method, and use alternatives for medical devices. An example aspiration catheter includes a catheter shaft having a proximal end region, a distal end region and a lumen extending therein. The aspiration catheter also includes a tip member having a length and a lumen extending therein, wherein the tip member is coupled to the distal end region of the catheter shaft and a guidewire shaft having a proximal end region and a distal end region. Further, the guidewire shaft extends within at least a portion of the lumen of the catheter shaft and the lumen of the tip member. Further, the distal end region of the guidewire shaft includes an interlock region and a portion of the tip member is engaged with at least a portion of the interlock region.

Alternatively or additionally to any of the examples above, wherein the interlock region includes a plurality of discrete slots.

Alternatively or additionally to any of the examples above, wherein each of the discrete slots includes an oval shape defined along an outer surface of the guidewire shaft.

Alternatively or additionally to any of the examples above, wherein each of the discrete slots extends radially inward from an outer surface of the guidewire shaft.

Alternatively or additionally to any of the examples above, wherein the plurality of discrete slots are aligned to form one or more rows of slots extending along a longitudinal axis of a wall of the guidewire shaft.

Alternatively or additionally to any of the examples above, wherein the plurality of discrete slots are aligned to form a first column of slots positioned adjacent a second column slots, and wherein the first column of slots is circumferentially offset from the second column of slots.

Alternatively or additionally to any of the examples above, wherein one or more of the discrete slots is oriented at an oblique angle relative to a longitudinal axis of a wall of the guidewire shaft.

Alternatively or additionally to any of the examples above, wherein a portion of the tip member is configured to extend into each of the discrete slots of the interlock region.

Alternatively or additionally to any of the examples above, wherein extension of the tip member into each of the discrete slots forms a mechanical bond between the tip member and the guidewire shaft.

Alternatively or additionally to any of the examples above, wherein the interlock region includes a length extending proximally from the distal end of the guidewire shaft, and wherein the length of the interlock region is substantially equal to the length of the tip member.

Alternatively or additionally to any of the examples above, wherein the interlock region includes a length extending proximally from the distal end of the guidewire shaft, and wherein the length of the interlock region is less than the length of the tip member.

Alternatively or additionally to any of the examples above, wherein a portion of the proximal end region of the guidewire shaft is attached to a manifold, and wherein at least a portion of the guidewire shaft extending between the manifold and the interlock region remains unattached to the catheter shaft.

Another example thrombectomy system includes a processor coupled to a pump and a thrombectomy catheter, wherein the thrombectomy catheter includes an outer shaft having a proximal end region attached to a manifold, a distal end region and a lumen extending therein. The thrombectomy catheter also includes a tip member having a length and a lumen extending therein, wherein the tip member is coupled to the distal end region of the outer shaft. The thrombectomy catheter also includes a guidewire shaft having a proximal end region and a distal end region. Further, the guidewire shaft extends within at least a portion of the lumen of the outer shaft and the lumen of the tip member. Further, the distal end region of the guidewire shaft includes an interlock region and a portion of the tip member is engaged with at least a portion of the interlock region of the guidewire shaft.

Alternatively or additionally to any of the examples above, wherein the interlock region includes a plurality of discrete slots.

Alternatively or additionally to any of the examples above, wherein each of the discrete slots includes an oval shape defined along an outer surface of the guidewire shaft.

Alternatively or additionally to any of the examples above, wherein the plurality of discrete slots are aligned to form a first column of slots positioned adjacent a second column slots, and wherein the first column of slots is circumferentially offset from the second column of slots.

Alternatively or additionally to any of the examples above, wherein each of the discrete slots extends radially inward from an outer surface of the guidewire shaft.

Alternatively or additionally to any of the examples above, wherein extension of the tip member into each of the discrete slots forms a mechanical bond between the tip member and the guidewire shaft.

Alternatively or additionally to any of the examples above, wherein at least a portion of the guidewire shaft extending between the manifold and the interlock region remains unattached to the outer shaft.

A method of manufacturing a thrombectomy catheter includes positioning an outer shaft adjacent a tip member, whereby a lumen of the outer shaft is in fluid communication with a lumen of the tip member. The method also includes positioning a guidewire shaft within the lumen of the outer shaft and the lumen of the tip member, wherein the guidewire shaft includes an interlock region having a plurality of discrete slots extending radially inward from an outer surface of the guidewire shaft. The method also includes aligning the guidewire shaft relative to the tip member such that a portion of the interlock region is positioned adjacent to the tip member. The method also includes reflowing the tip member such that a portion of the tip member extends into each of the discrete slots of the interlock region.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of an example thrombectomy system;

FIG. 2 is a partially exploded perspective view of a portion of the thrombectomy system of FIG. 1;

FIG. 3 is a longitudinal cross-sectional view of a distal end region of an example thrombectomy catheter;

FIG. 4 is a partially exploded perspective view of the distal end region of the thrombectomy catheter show in FIG. 3 in an example manufacturing step;

FIG. 5 is detailed view of a portion of the thrombectomy catheter of FIG. 3;

FIG. 6 is an embodiment of a portion of the thrombectomy catheter of FIG. 3;

FIG. 7 is an embodiment of a portion of the thrombectomy catheter of FIG. 3;

FIG. 8 is an embodiment of a portion of the thrombectomy catheter of FIG. 3;

FIG. 9 is a cross-sectional view of the distal end region of the thrombectomy catheter show in FIG. 3 in an example manufacturing step;

FIG. 10A is a detailed view of a portion of the thrombectomy catheter shown in FIG. 9;

FIG. 10B is another embodiment of the detailed view shown in FIG. 9;

FIG. 10C is another embodiment of the detailed view shown in FIG. 9;

FIG. 11 is a cross-sectional view of a portion of the distal end region of the thrombectomy catheter in an example manufacturing step;

FIG. 12 is a detailed view of a portion of the thrombectomy catheter shown in FIG. 3.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Thrombectomy catheters and systems may be used to remove thrombus, plaques, lesions, clots, etc. from veins or arteries. Some thrombectomy catheters may utilize high velocity saline jets in a series to entrain fluid or clot material into and through the shaft of the catheter. Other thrombectomy systems may utilize one or more pressurized saline jets which travel backwards to create a low-pressure zone and a vacuum effect, whereby the vacuum pulls clot material into and through the distal tip and shaft of the catheter. However, prolonged operation of a thrombectomy system may create increased forces placed on the distal tip of the thrombectomy catheter. Accordingly, it may be desirable to design a thrombectomy catheter which includes a strengthened distal tip bond configured to resist increased forces placed thereon. Thrombectomy systems which include a thrombectomy catheter having a strengthened distal tip bond configured to resist increased forces placed thereon are disclosed herein.

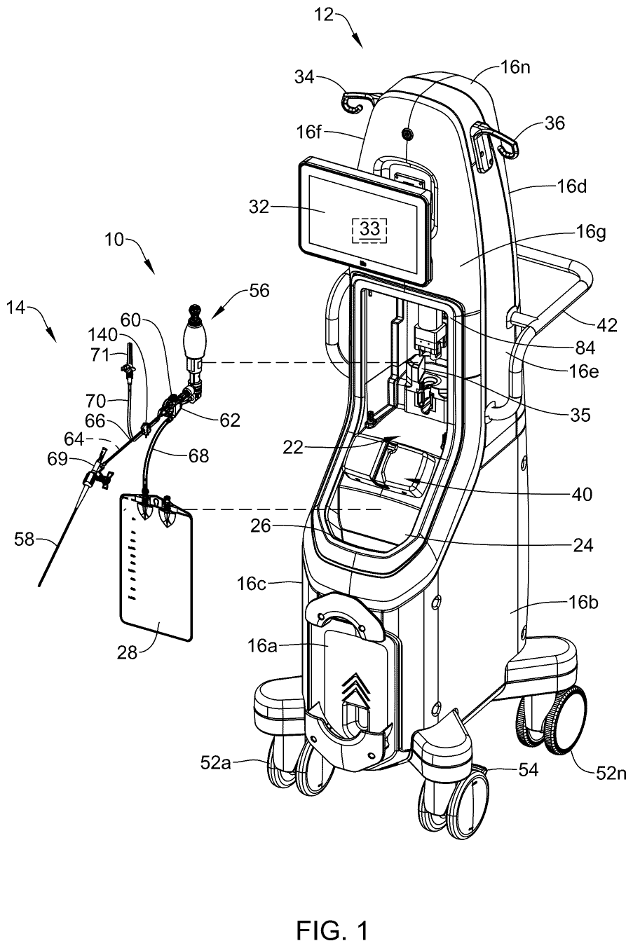

FIG. 1 is a perspective view of an illustrative thrombectomy system 10. The thrombectomy system 10 may include a control console or drive unit 12 and a pump/catheter assembly 14. In some instances, the pump/catheter assembly 14 may be a single use device in which a new pump/catheter assembly 14 may be used with the drive unit 12 for each medical procedure. Shown on the drive unit 12 are a plurality of removable panels 16a-16n about and along the drive unit 12 enclosing the internal structure of the 25 drive unit 12. An illustrative drive unit 12 is described in commonly assigned U.S. Pat. No. 7,935,077, titled THROMBECTOMY CATHETER DEPLOYMENT SYSTEM, the disclosure of which is hereby incorporated by reference. Centrally located in the drive unit 12 and aligned to the lower region of the panel 16g may be automatically opening doors 18 and 20 which open to expose the interior of the drive unit 12 to provide access to a carriage assembly 22. The carriage assembly 22, which may accommodate components of the pump/catheter assembly 14, as discussed further herein, is shown accessible via opening the closed doors 18 and 20. The drive unit 12 may include a catch basin for collecting fluid leakage from the components of the pump/catheter assembly 14. For example, a removable drip tray 24 is shown located on the front of the drive unit 12 extending from below the carriage assembly 22 toward the panel 16a. Other configurations of catch basins are also contemplated. The drip tray 24 and a removable receptacle 26 may collectively support and accommodate an effluent collection bag, such as effluent collection bag 28 of the pump/catheter assembly 14. In other instances, the drive unit 12 may include a different structure, such as a hook for hanging the effluent collection bag 28 from, or a shelf for setting the effluent collection bag 28 on. In instances where the carriage assembly 22 is movable, a carriage assembly activation switch 30 may be provided with the drive unit 12, such as located on panel 16g, to selectively position the carriage assembly 22 inwardly or outwardly. A user interface 32, including memory capabilities, may be provided with the drive unit 12, such as located at the upper region of the drive unit 12 between the upper regions of the upper side panels 16e and 16f. Saline bag hooks 34 and 36 may extend through the panels 16e and 16f to hang saline bags therefrom. The drive unit 12 may include a handle 42 as well as a plurality of wheels 52a-52n and brake pedals 54 for wheel lockage to assist in maneuvering the drive unit 12 by medical personnel.

The pump/catheter assembly 14, which may be a disposable single-use device, is shown unattached from the drive unit 12. The pump/catheter assembly 14 includes a pump 56 and a thrombectomy catheter 58. During use, a portion of the pump/catheter assembly 14 may be secured within a portion of the drive unit 12. Other components included in the pump/catheter assembly 14 may include a bubble trap 60 attached to the pump 56, a connection manifold assembly 62 connected to the bubble trap 60, an effluent return tube 66 connected between the connection manifold assembly 62 and the thrombectomy catheter 58, a high-pressure fluid supply tube 64 attached between the output of the pump 56 and the thrombectomy catheter 58 which may be coaxially arranged inside the effluent return tube 66, a catheter manifold 69 (between the distal end of the effluent return tube 66 and the proximal end of the thrombectomy catheter 58, an effluent waste tube 68 connecting the effluent collection bag 28 to the connection manifold assembly 62, and a fluid supply tube 70 having a bag spike 71 connecting a fluid supply bag 72 (e.g., a saline bag) to the connection manifold assembly 62. The fluid supply tube 70 may be in fluid communication with the interior of the bubble trap 60 to provide fluid from the fluid supply bag 72 to the pump 56 and then to the thrombectomy catheter 58 through the high-pressure fluid supply tube 64.

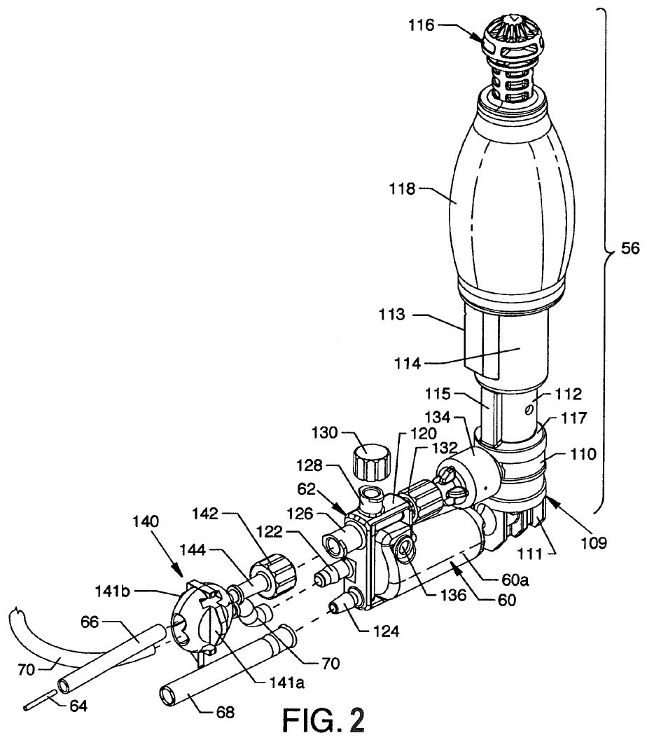

FIG. 2 is a partially exploded perspective view of several components of the pump/catheter assembly 14 generally including the pump 56, the bubble trap 60, the connection manifold assembly 62, and a fixture 140. The pump 56 centers about a tubular body 112. Components are located about the lower region of the tubular body 112 and include a base 109 having an upper portion 110 and a lower portion 111 both positioned about the lower region of the tubular body 112. An annular surface 117 is included at the top of the upper portion 110 of the base 109 for intimate contact with capture tabs of the carriage assembly 22 to contain the pump 56 within the carriage assembly 22. A top body 114, is positioned about the upper region of the tubular body 112. The base 109 and the top body 114, as well as a connecting panel 115, may be molded or otherwise suitably constructed to encompass the greater part of the tubular body 112, for example. A data plate 113 may also be included on the top body 114 for the inclusion of a barcode, an RFID tag, or other informational displays to determine operational parameters of the device.

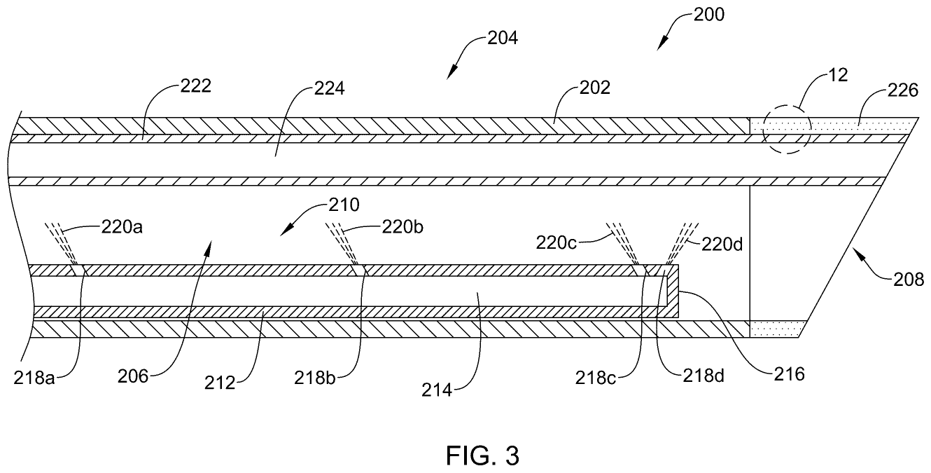

The pump 56 may include a hemispherically-shaped pump piston head 116 having a flexible boot 118 connected to and extending between the top body 114 and the pump piston head 116. In some instances, the geometrically configured lower portion 111 of the base 109 may serve as a mount for one end of the bubble trap 60 (FIG. 3).

The connection manifold assembly 62 may be secured directly to the other end of the bubble trap 60 and in some instances may include a bracket 120 to which is attached a vertically oriented tubular manifold 148 having a plurality of ports attached or formed therethrough including a fluid (e.g., saline) inlet port 122, an effluent outlet port 124, a Luer style effluent return port 126, and/or an auxiliary port 128 and cap 130. Also shown are connectors 132 and 134 connecting extending between the connection manifold assembly 62 and the upper portion 110 of the base 109.

The bubble trap 60 may include mating halves of which one mating half 60a is shown. A hydrophobic filter 136 may be included at the upper forward region of the bubble trap half 60a. Another hydrophobic filter may be included on the second bubble trap half (not explicitly shown) which opposes the hydrophobic filter 136 on the bubble trap half 60a.

The fixture 140, and components associated therewith, assists in support and connection of the effluent return tube 66 to the effluent return port 126 by a connector 142 combined continuously with a connection tube 144, and also assists in support, passage and connection of the fluid supply tube 70 with the fluid inlet port 122. The fixture 140 may include outwardly extending vertically aligned and opposed tabs 141a and 141b which prevent the fixture 140 and associated effluent return tube 66 containing the high-pressure fluid supply tube 64 and the fluid supply tube 70 from contacting a roller pump (not explicitly shown) provided with the drive unit 12, such as located in the carriage assembly 22 or adjacent thereto.

FIG. 3 is a cross-sectional view of a distal end region 204 of another illustrative thrombectomy catheter 200. The thrombectomy catheter 200 may be one illustrative example of the thrombectomy catheter 58 described above. The thrombectomy catheter 200 may include a tubular member or catheter shaft 202 extending from a proximal end region (not explicitly shown) configured to remain outside the shaft to a distal end region 204. The catheter shaft 202 may be one illustrative example of, or be in fluid communication with, the effluent return tube 66 of the thrombectomy catheter 58 described above. A lumen 206 may extend from the proximal end region to the distal end region 204 of the catheter shaft 202. The catheter shaft 202 may terminate at a distally facing distal opening 208 at the distal end of the catheter shaft 202. In some instances, the distal opening 208 may be in a plane that extends generally orthogonal to a longitudinal axis of the catheter shaft 202. In other instances, the distal opening 208 may be in a plane that extends generally oblique to a longitudinal axis of the catheter shaft 202. In other words, in some examples, the distal end of the thrombectomy catheter 200 may be tapered relative to the longitudinal axis of the catheter shaft 202. Generally, the distal opening 208 may be an entrainment inflow orifice. While not explicitly shown, the catheter shaft 202 may include one or more markers (e.g., radiopaque marker bands) disposed along the catheter shaft 202. Further, while not explicitly shown, in some embodiments, the catheter shaft 202 may include one or more openings extending through a sidewall thereof, if desired.

The thrombectomy catheter 200 may further include a high-pressure fluid supply tube 210. The high-pressure fluid supply tube 210 may be one illustrative example of, or be in fluid communication with, the high-pressure fluid supply tube 66 of the thrombectomy catheter 58 described above. The high-pressure fluid supply tube 210 may be disposed within and extend through the lumen 206 of the catheter shaft 202. The high-pressure fluid supply tube 210 may include a supply tube wall 212 defining a lumen or fluid pathway 214 extending therethrough. In at least some instances, the high-pressure fluid supply tube 210 may have a closed distal end 216. Because of this, fluid may be able to pass distally through the fluid pathway 214 but does not exit the distal end. The high-pressure fluid supply tube 210 may extend along a length of the catheter shaft 202 with the distal end 216 located within the lumen 206 of the catheter shaft 202 proximal to the distal opening 208 at the distal end of the catheter shaft 202. A proximal end of the high-pressure fluid supply tube 210 may be in fluid communication with the pump 56 described herein, to provide high-pressure fluid to the fluid pathway 214 of the high-pressure fluid supply tube 210.

A plurality of jet orifices 218a-d (collectively, 218) may be defined along the supply tube wall 212. For example, the supply tube wall 212 may include two, three, four, five, six, or more jet orifices 218. The jet orifices 218 may be spaced along the supply tube wall 212 at any desired intervals. For example, each of the jet orifices 218 may be equidistantly spaced from adjacent jet orifices 218 along the length of the supply tube wall 212. In other instances, the jet orifices 218 may be arranged such that the spacing between adjacent jet orifices 218 near the distal end of the supply tube wall 212 is closer than the spacing between adjacent jet orifices 218 near the proximal end of the supply tube wall 212. For instance, the spacing between the orifices 218 may gradually increase as you move proximally along the length of the shaft, or the spacing may increase in a stepwise configuration. In some instances, some or all of the jet orifices 218 may be axially aligned along the supply tube wall 212. In other instances, one or more of the jet orifices 218 may be circumferentially offset from one another about the supply tube wall 212. A number of patterns are contemplated including a helical pattern, a pattern where no two jet orifices 218 are disposed at the same axial location, a regular pattern including two or more jet orifices 218 disposed at the same axial location, an irregular pattern (where some of the jet orifices 218 may or may not be disposed at the same axial location), etc.

The jet orifices 218 may be formed using a suitable method such as electron discharge machining, etching, cutting (e.g., including laser cutting), or the like. In some instances, one or more of the jet orifices 218 may have a substantially round shape. In other instances, one or more of the jet orifices 218 may have a substantially non-round shape (e.g., oval, polygonal, irregular, etc.). In some instances, the jet orifices 218 may be beveled or otherwise include a beveled surface. It is contemplated that a size and/or a shape of the jet orifices 218 may be varied to vary the velocity of the fluid exiting the jet orifices. For example, decreasing the size of the jet orifices 218 may increase the velocity of the fluid exiting the jet orifices 218. In some embodiments, the size of the jet orifices 218 may be varied based on the pressure capacity of the thrombectomy system, the number of jet orifices, the dimensions of the high-pressure fluid supply tube 210 (e.g., length, wall thickness, inner diameter, etc.), and/or combinations thereof. In some examples, the jet orifices 218 may have a cross-sectional dimension in the range of about 0.0018″ (0.0018 inches) to about 0.0022″. However, the jet orifices 218 can have a cross-sectional dimension of less than 0.0018″ or greater than 0.0022″, as desired.

Infusion of motive fluid through the lumen 214 of the supply tube wall 212 may result in fluid being jetted through the jet orifices 218 and the generation of a proximally directed aspiration force. At least some of the jet orifices 218a-c may be angled in a proximal direction or otherwise designed to infuse fluid (e.g., a motive fluid, a liquid, a gas or air, steam, a fluid with particles disposed therein, or the like) through the jet orifices 218a-c and into the lumen 206 of the catheter shaft 202 in a generally proximal direction as depicted by lines 220a-c representing motive jetted fluid projecting generally proximally from the jet orifices 218a-c. For example, each of the jet orifices 218a-c may be arranged at an acute angle to the longitudinal axis of the supply tube wall 212 such that the jet orifices 218a-c angle in a proximal direction. In some embodiments, one or more of the jet orifices 218d may be designed to infuse fluid (e.g., a motive fluid, a liquid, a gas or air, steam, a fluid with particles disposed therein, or the like) through the jet orifice(s) 218d and into the lumen 206 of the catheter shaft 202 in a generally distal direction as depicted by lines 220d representing motive jetted fluid projecting generally distally from the jet orifice 218d. For example, the jet orifice 218d may be arranged at an oblique angle to the longitudinal axis of the supply tube wall 212 such that the jet orifice 218d angles in a distal direction. It is contemplated that an angle of the jet orifices 218 and thus the motive jetted fluid 220 may be varied to adjust the velocity of the fluid exiting the jet orifices 218. As further described herein, the supply tube wall 212 may include one or more, or a plurality of proximally oriented or directed jet orifices 218a, 218b, 218c (i.e., jet orifices configured to direct fluid infused through the lumen 214 of the supply tube wall 212 in a proximal direction) and the supply tube wall 212 may include one or more, or a plurality of distally oriented or directed jet orifices 218d (i.e., jet orifices configured to direct fluid infused through the lumen 214 of the supply tube wall 212 in a distal direction). In some examples, the distally projecting jet orifice 218d may be axially aligned with one or more of the proximally projecting jet orifices 218a-c. In other examples, the distally projecting jet orifice 218d may be circumferentially offset from one or more of the proximally projecting jet orifices 218a-c. For example, the distally projecting jet orifice 218d may be circumferentially offset from one or more of the proximally projecting jet orifices 218a-c by in the range of about 10° to about 350° or about 45° to about 135°.

The distally projecting jet orifice 218d may be the distalmost jet orifice, with the proximally projecting jet orifices 218a-c positioned proximal of the distally projecting jet orifice 218d. However, this is not required. In some embodiments, the distally projecting jet orifice 218d may be positioned proximal to at least one proximally projecting jet orifice 218a-c. While the supply tube wall 212 is illustrated as including only a single distally projecting jet orifice 218d, the supply tube wall 212 may include more than one distally projecting jet orifice, as desired. When more than one distally projecting jet orifice 218d is provided, the distally projecting jet orifices may be positioned at differing axial and/or circumferential locations from one another or similar axial and/or circumferential locations as one another, as desired. The distally projecting jet orifice(s) 218d may break up particles as they are drawn into the lumen 206 of the catheter shaft 202 while the proximally projecting jet orifices 218a-c may move particles proximally along the catheter shaft 202.

The performance of the thrombectomy catheter 200 and the high-pressure fluid supply tube 210 may be directly related to the velocity of the motive jetted fluid 220 exiting the jet orifices 218 and the shear-induced turbulent flux created by the jetted motive fluid 220. For example, the more powerful the jetted motive fluid 220, the higher the aspiration rates may be. It is further contemplated that the performance of the jet-powered aspiration catheter 200 may be directly related to the speed at which the clot can be entrained into the catheter 200, macerated, and removed from the shaft. Any clogging that occurs within the catheter shaft 202 may reduce or completely stop the removal of the clot. The addition of the distally projecting jet orifice 218d may macerate any clot that enters the distal opening 208 of the catheter shaft 202 thus helping prevent clogging. For example, at the point of impingement of the distally oriented motive jetted fluid 220d the motive jetted fluid 220d may deflect distally creating flow out the tip of the distal opening 208 of the catheter shaft 202, effectively macerating any clot that enters the tip of the device and eliminating or reducing risk of the distal opening 208 of the catheter shaft 202 becoming blocked or clogged. It is contemplated that the properties (size, shape, angle, number, spacing, etc.) of the jet orifices 218 may be varied to obtain a fluid velocity that creates an optimum de-clogging effect without hindering the proximal flow of a clot within the lumen 206 of the catheter shaft 202 or the clot evacuation rate.

The distally projecting jet orifice 218d may be proximally spaced a distance from the distal opening 208 of the catheter shaft 202. It is contemplated that the longitudinal location of the distally projecting jet orifice 218d on the supply tube wall 212 and relative to the distal opening 208 of the catheter shaft 202 may be varied based on a size of the aperture of the distally projecting jet orifice 218d, the velocity of the fluid within the lumen 214 of the supply tube wall 212, the angle of the distally projecting jet orifice 218d, or combinations thereof, etc. to ensure the distally oriented motive jetted fluid 220d impinges the inner surface of the catheter shaft 202. In one illustrative example, the distally projecting jet orifice 218d may be positioned such that the distally oriented motive jetted fluid 220d impinges an inner surface of the catheter shaft 202 such that the distally oriented motive jetted fluid 220d does not damage the vessel. For example, the distally projecting jet orifice 218d may be positioned such that the distally oriented motive jetted fluid 220d impinges an inner surface of the catheter shaft 202 in the range of about 0.070″ to about 0.090″ proximal to the distal end of the catheter shaft 202. This is just one example. The impingement location of the motive jetted fluid 220d of the distally projecting jet orifice 218d may be less than 0.070″ or more than 0.090″ proximal to the distal end of the catheter shaft 202, as desired.

In some instances, the jet orifices 218 may be oriented at an angle relative to the longitudinal axis of the supply tube wall 212. For example, the proximally projecting jet orifices 218a-c may be oriented at an oblique (e.g., acute) angle relative to the longitudinal axis of the supply tube wall 212 and/or oriented at an angle greater than zero degrees and less than ninety degrees relative to the longitudinal axis of the supply tube wall 212. It is contemplated that a distally projecting jet orifice 218d may be oriented at an oblique (e.g., obtuse) angle relative to the longitudinal axis of the supply tube wall 212 and/or oriented at an angle greater than 90 degrees and less than 180 degrees relative to the longitudinal axis of the supply tube wall 212. In other instances, the jet orifices 218 may be oriented perpendicular to the longitudinal axis of the supply tube wall 212 (e.g., at an angle of about 90 degrees relative to the longitudinal axis of the supply tube wall 212). The angle may or may not be the same for all the jet orifices 218.

In at least some instances, the jet orifices 218 may be understood as being arranged in series. In other words, the jet orifices 218 may be arranged such that adjacent jet orifices 218 are spaced longitudinally apart at various locations along the longitudinal axis of the supply tube wall 212. For example, the jet orifices 218 may be uniformly or non-uniformly spaced along of a length of the supply tube wall 212. This may position the jet orifices 218 at axially spaced apart locations within the catheter shaft 202 and along the length thereof. For example, the jet orifices 218 may be spaced along an entire length of the supply tube wall 212 and correspondingly along an entire length of the catheter shaft 202, or portions thereof, as desired. In some examples, the jet orifices 218 may be spaced at intervals in the range of every 5 inches to every 15, or in the range of every 6 inches to every 12 inches along a length of the supply tube wall 212. In other instances, the spacing between the jet orifices 218 may be less than every 5 inches or greater than every 15 inches. Accordingly, motive fluid leaves via the jet orifices 218 forming a jetted motive fluid 220a-d (collectively, 420). This jetted motive fluid 220 enters an entrainment material where the shear layer between the two causes turbulence, mixing, and transfer of momentum. Entrainment material may enter the distal opening 208 and then may be urged proximally by momentum transfer. As the mixture of jetted motive fluid 220 and entrainment material migrates proximally, the material may sequentially approach a number of jet orifices 218. Upon interaction with the jetted motive fluid 220 from each individual jet orifice 218, the momentum in the entrainment material mixture may increase, and the thrombogenic material may more readily flow proximally through the catheter shaft 202 for removal. The increase in momentum may allow for the catheter shaft 202 to be used without a second or outflow orifice (e.g., positioned proximally of the distal opening 208). Alternatively, some of the entrapped thrombogenic material may exit the catheter shaft 202 through a second orifice (not shown), e.g., in a sidewall of the catheter shaft 202, positioned proximal to the distal opening 208, recirculate to the distal opening 208 (e.g., one or more times), and then move proximally through the lumen 206 of the catheter shaft 202.

It is further contemplated that the distally oriented motive jetted fluid 220d may be partially to fully entrained by the force generated by the proximally oriented motive jetted fluid 220a-c. When the clot/thrombus reaches the distally oriented motive jetted fluid 220d, the shear stress may masticate the clot/thrombus. It is contemplated that when the distal opening 208 of the catheter shaft 202 is sealed with a clot/thrombus, the force generated by the proximally oriented motive jetted fluid 220a-c may be transferred to the surface of the clot/thrombus in a proximal direction. As a result, the distally oriented motive jetted fluid 220d may no longer be entrained and may transfer force in the distal direction to the surface of the clot/thrombus. Thus, when the distal opening 208 is clogged or plugged, an extreme shear mechanism of action is created where the distal and proximal force vectors combine together to focus all of the shear stress to the surface of the clot/thrombus to masticate the clot/thrombus and unplug the distal opening 208. It is contemplated that the shear stress on the clot/thrombus may be much larger in magnitude when the distally oriented motive jetted fluid 220d is at a smaller angle (e.g., closer to 180 degrees relative to the longitudinal axis of the supply tube wall 212 than to orthogonal to the longitudinal axis of the supply tube wall 212).

FIG. 3 illustrates that the thrombectomy catheter 200 may further include a tip member 226 positioned along the distal end region 204. Additionally, FIG. 3 illustrates that the thrombectomy may also include a guidewire shaft 222. The guidewire shaft 222 may extend within the lumen 206 of the catheter shaft 202. In some examples, a proximal end of the guidewire shaft 222 may be coupled (e.g., attached, bonded) to the catheter manifold 69 (shown in FIG. 1). Additionally, as will be discussed in greater detail herein, the distal end region of the guidewire shaft 222 may be bonded to the tip member 226. It can be appreciated that, in some examples, the portion of the guidewire shaft 222 extending between the catheter manifold 69 and the tip member 226 may remain attached to the catheter shaft 202. For example, a proximal portion of the guidewire shaft 222 may be attached to the catheter manifold 69, a distal portion of the guidewire shaft 222 may be attached to the tip member 226 and the portion of the guidewire shaft 222 extending between the catheter manifold 69 and the tip member 226 may be unattached and free to move within the lumen 206 of the catheter shaft 202. Further, FIG. 3 illustrates that the guidewire shaft 222 may include a lumen 224 configured to permit a guidewire to extend within.

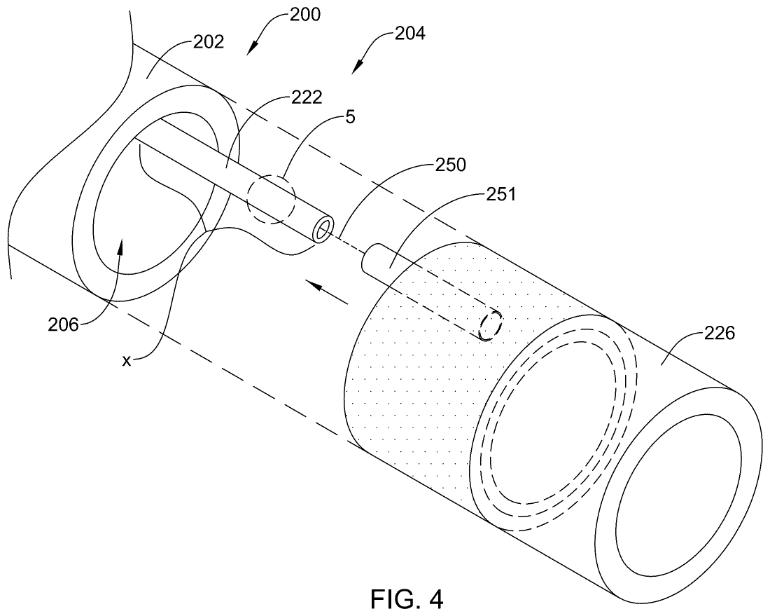

FIG. 4 illustrates example manufacturing steps for the thrombectomy catheter 200 described herein. FIG. 4 illustrates that an initial manufacturing step may include positioning a bonding sleeve 251 over top a portion of the distal end region guidewire shaft 222 and subsequently attaching the bonding sleeve 251 to the guidewire shaft 222. After the bonding sleeve 251 is attached to the distal end region of the guidewire shaft 222, FIG. 4 illustrates that the tip member 226 may be positioned over top a portion of the bonding sleeve 251 (which has been attached to distal end region guidewire shaft 222) and subsequently attaching the tip member 226 to the bonding sleeve 251 and, consequently, the guidewire shaft 222.

In some examples, the bonding sleeve 251 and/or the tip member 226 may be formed from a polymer material including, but not limited to a thermoplastic polymer. Other suitable polymers which may be utilized to form the tip member 226 may include Vestamid®, Grilamid®, polyamides including Nylon 6, Nylon 66, Nylon 11, Nylon 12, polyether block amide copolymer including 32D Pebax®, 35D Pebax®, 48D Pebax®, 55D Pebax®, 68D Pebax®, 72D Pebax®, Pebax® MED, Rilsan® MED, Rilsamid® MED, Rilsan® Clear MED and Kynar® MED. A non-limiting list of examples which may be utilized to form the bonding sleeve 251 and the tip member 226 is disclosed below.

Further, it can be appreciated that the catheter shaft 202 may be formed from a polymer material. For example, the catheter shaft 202 may be formed from a polymer material including, but not limited to a thermoplastic polymer (e.g., Pebax®). Other suitable polymers which may be utilized to form the tip member 226 may include Vestamid®, Grilamid®, polyamides including Nylon 6, Nylon 66, Nylon 11, Nylon 12, polyether block amide copolymer including 32D Pebax®, 35D Pebax®, 48D Pebax®, 55D Pebax®, 68D Pebax®, 72D Pebax®, Pebax® MED, Rilsan® MED, Rilsamid® MED, Rilsan® Clear MED and Kynar® MED. A non-limiting list of examples which may be utilized to form the catheter shaft 202 is disclosed below.

The guidewire shaft 222 may be formed from a polymer material including, but not limited to a polyimide. Other suitable polymers which may be utilized to form the guidewire shaft 222 may include PEEK (polyether ether ketone). A non-limiting list of examples which may be utilized to form the guidewire shaft 222 is disclosed below.

It can be appreciated that constructing the guidewire shaft 222 from a thermoset polyimide provides sufficient strength to the guidewire shaft 222, thereby allowing the guidewire shaft 222 to withstand forces of the jetted motive fluid 220a-d leaving the jet orifices 218a-d. However, it can be further appreciated that the thermoset polyimide material utilized to form the guidewire shaft 222 may not be capable of forming a chemical bond to the thermoplastic polymer used to form the catheter shaft 202 and the tip member 226. Accordingly, it may be desirable to design a portion of the guidewire shaft 222 to include one or more features which permit the thermoset polyimide material utilized to form the guidewire shaft 222 to form a mechanical bond to the thermoplastic polymer used to form the tip member 226.

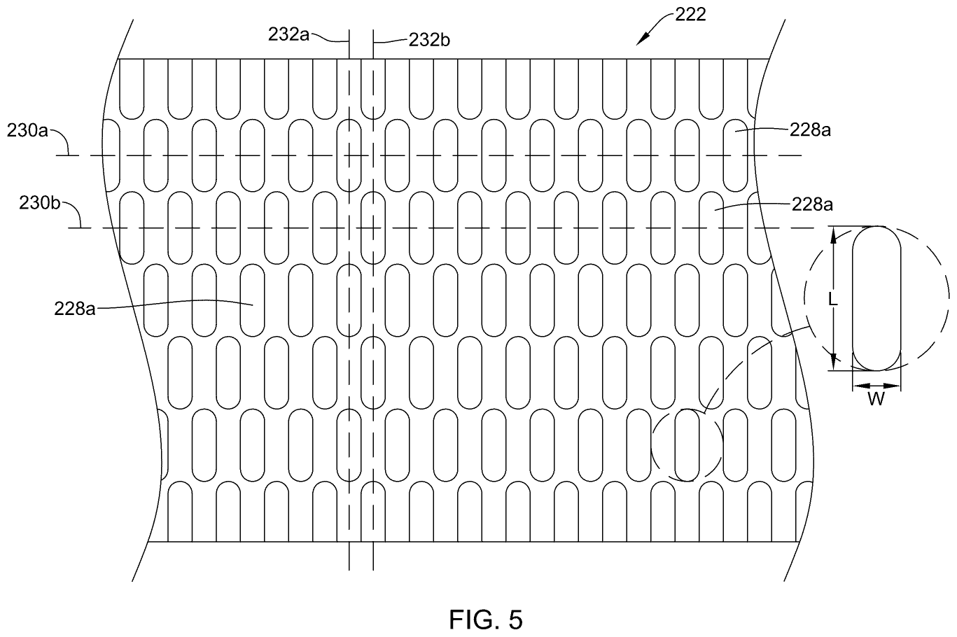

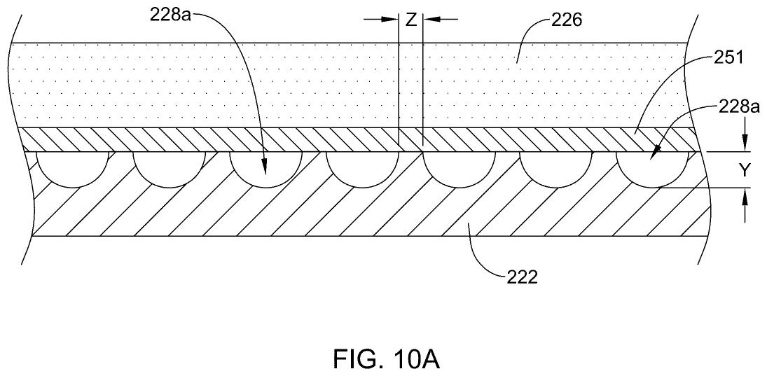

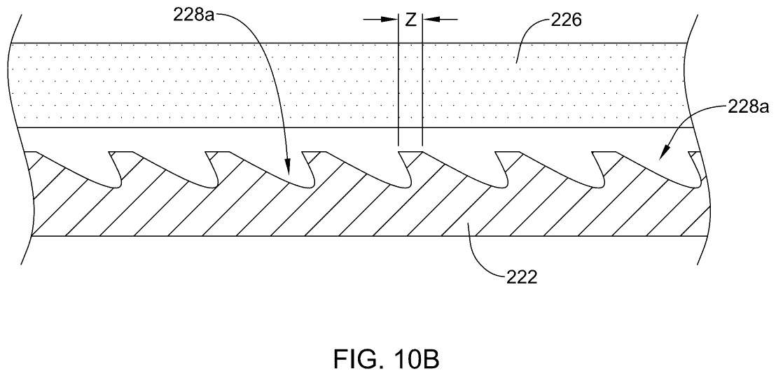

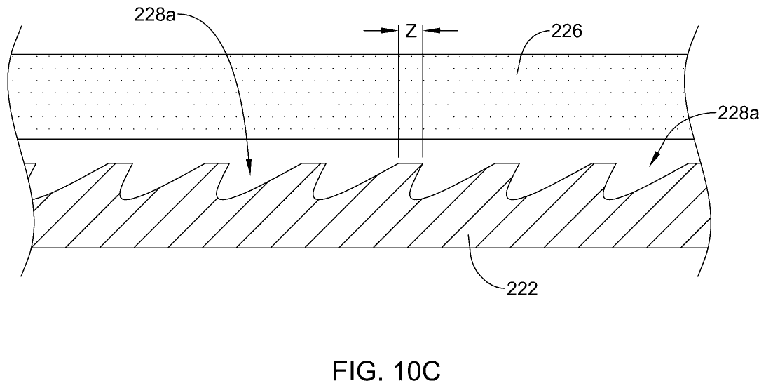

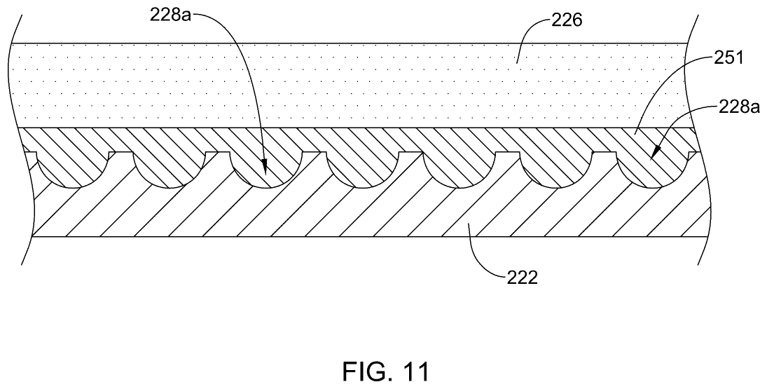

FIG. 5 illustrates a detailed view of a portion of the distal end region of the guidewire shaft 222 of FIG. 4. FIG. 5 illustrates that the distal end region of the guidewire shaft 222 may include an interlock pattern (e.g., tip engagement pattern, slotted pattern, micro-slotted pattern, etched pattern, laser-etched pattern, etc.) formed from a plurality of individual slots (e.g., grooves, channels, cavities, depressions, etc.) 228a having a length L, a width W and depth extending into the wall of the guidewire shaft 222 (the depth Y of the individual slots 228a is shown in FIGS. 10-11). FIG. 5 illustrates that each of the individual slots 228a may include a general oval shape. However, this is not intended to be limiting. Rather, it can be appreciated that, in some examples, each of the individual slots 228a may include a variety of shapes including circular, rectangular, square, triangular, polygonal, star, dog bone, combinations thereof and/or any other suitable geometric shape. It can be appreciated that the plurality of slots 228a forming the interlock pattern may be formed from a variety of techniques including, but not limited to utilizing a femtosecond laser to cut out each individual slot 228a.

Further, FIG. 5 illustrates that the length L of each individual slot 228a forming the interlock pattern shown in FIG. 5 is oriented generally perpendicular to the longitudinal axis 250 of the wall of the guidewire shaft 222. Further, the individual slots 228a forming the interlock pattern shown in FIG. 5 may be aligned in one or more “rows”, wherein the row of individual slots 228a extends generally parallel to the longitudinal axis 250 of the wall of the guidewire shaft 222. For example, FIG. 5 illustrates a plurality of individual slots 228a aligned along a first row 230a and a plurality of individual slots aligned along a second row 230b.

Further, FIG. 5 illustrates that, in some examples, a plurality of individual slots 228a may be aligned circumferentially to form one or more columns, whereby the longitudinal axis of each slot 228a extends along the circumference of the guidewire shaft 222. Further, in some examples, adjacent columns of slots 228a may be circumferentially offset from one another along the longitudinal axis 250 of the wall of the guidewire shaft 222. For example, FIG. 5 illustrates a plurality of individual slots 228a aligned along a first column 232a and a plurality of individual slots aligned along a second column 232b, whereby the first column is circumferentially offset from the second column 232b. In some examples, the interlock pattern may include about 150 to about 250 columns of slots 228a, or about 175 to about 225 columns of slots 228a, or about 185 to about 215 columns of slots 228a, or about 200 columns of slots 228a extending along the longitudinal axis 250 of the wall of the guidewire shaft 222.

Further, in some examples, the interlock pattern may include about 2, 3, 4, 5, 6, 7, 8, 9, 10, 11, 12 or more individual slots 228a circumferentially spaced around the circumference of the guidewire shaft 222. However, this is not intended to be limiting. Rather, it is contemplated that, in some examples, the individual slots 228a may not extend completely around the circumference of the guidewire shaft 222. For example, the plurality of slots 228a may extend partially around the circumference of the guidewire shaft 222. In some examples, the plurality of slots 228a may extend about a quarter of the way around the circumference of the guidewire shaft 222. In other examples, the plurality of slots 228a may extend about halfway around the circumference of the guidewire shaft 222. In other examples, the plurality of slots 228a may extend about three-quarters around the circumference of the guidewire shaft 222. In yet other examples, the plurality of slots 228a may extend in a spiral pattern around the circumference of the guidewire shaft 222.

The detailed view of FIG. 5 illustrates that each individual slot 228a may include a length L of about 0.0005″ to about 0.0075″, or about 0.0010″ to about 0.0060″, or about 0.0015″ to about 0.0050″, or about 0.0020″ to about 0.0040″, or about 0.0025″ to about 0.0035″, or about 0.0033″. Additionally, the detailed view of FIG. 5 illustrates that, in some examples, each individual slot 228a may include a width W of about 0.000090″ to about 0.0020″, or about 0.000095″ to about 0.0015″, or about 0.0001″ to about 0.0012″, or about 0.0005″ to about 0.0010″, or about 0.0008″.

Referring to FIG. 4, it can be appreciated that the interlock pattern formed along the distal end region of the guidewire shaft 222 may extend proximally from the distal end of the guidewire shaft 222 a distance X. In some examples, the distance X may be substantially equivalent to the length of the tip member 226. For example, the length X may be about 0.060″ to about 0.140″, or about 0.070″ to about 0.130″, or about 0.080″ to about 0.120″, or about 0.090″ to about 0.110″, or about 0.095″ to about 0.105″, or about 0.100″. In other words, the interlock pattern formed along the distal end region of the guidewire shaft 222 may extend along the entire length of the tip member 226. In other examples, the interlock pattern may consist of a single slot 228a or single ring of slots 228a extending circumferentially around the outer surface of the guidewire shaft 222. In other examples, the interlock pattern may extend along the entire length of the guidewire shaft 222. In yet other examples, the interlock pattern may extend along any portion of the guidewire shaft 222 including portions of the guidewire shaft 222 positioned underneath the catheter shaft 202. Portions of the guidewire shaft 222 including the interlock pattern which is positioned underneath the catheter shaft 202 may be attached to the catheter shaft 202.

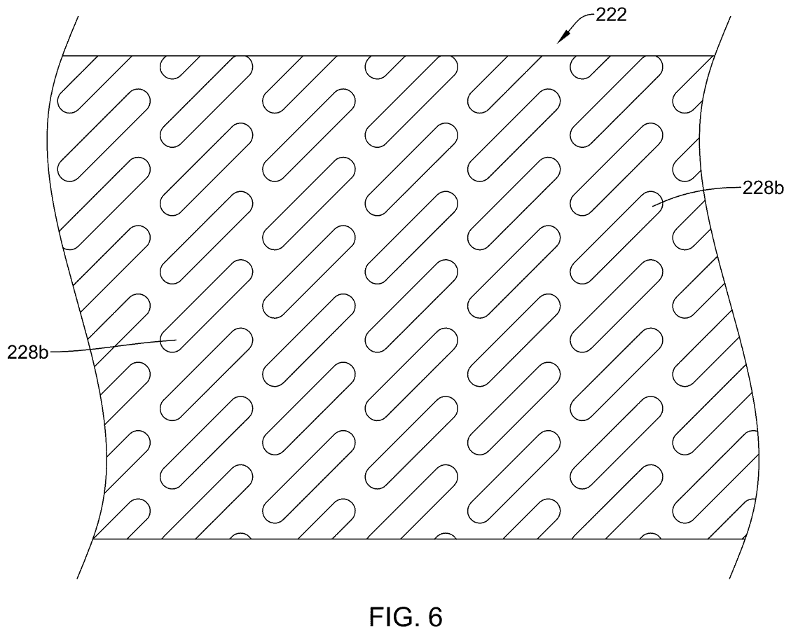

FIGS. 6-8 illustrates example embodiments of different interlock patterns which function similar to the interlock pattern described herein with respect to FIG. 5.

FIG. 6 illustrates an example interlock pattern including a plurality of individual slots 228b formed along the guidewire shaft 222. It can be appreciated that the slots 228b may be similar in form and function to the slots 228a described herein. However, FIG. 6 illustrates that each individual slot 228b may be oriented an at oblique (e.g., acute) angle relative to the longitudinal axis 250 of the wall of the guidewire shaft 222.

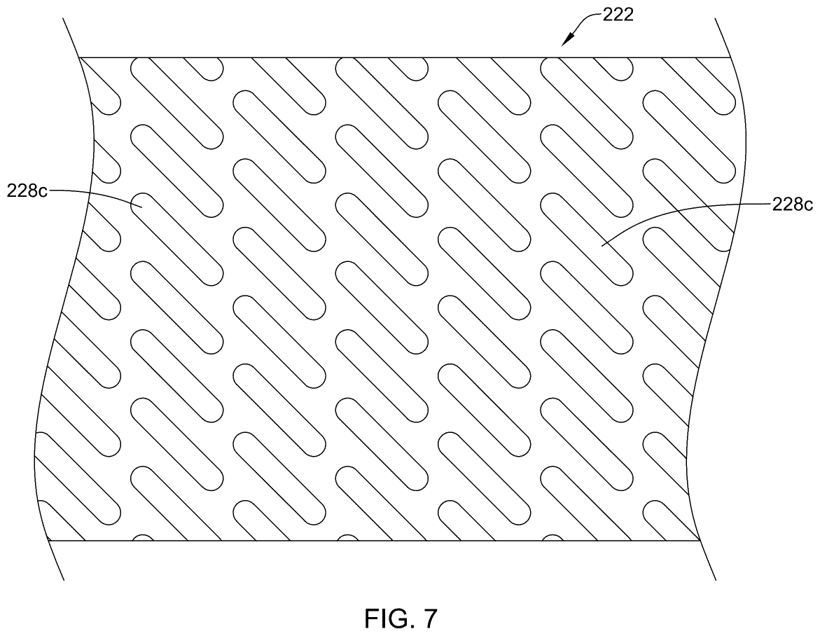

FIG. 7 illustrates another example interlock pattern including a plurality of individual slots 228c formed along the guidewire shaft 222. It can be appreciated that the slots 228c may be similar in form and function to the slots 228a described herein. However, FIG. 7 illustrates that each individual slot 228c may be oriented an at oblique (e.g., obtuse) angle relative to the longitudinal axis 250 of the guidewire shaft 222.

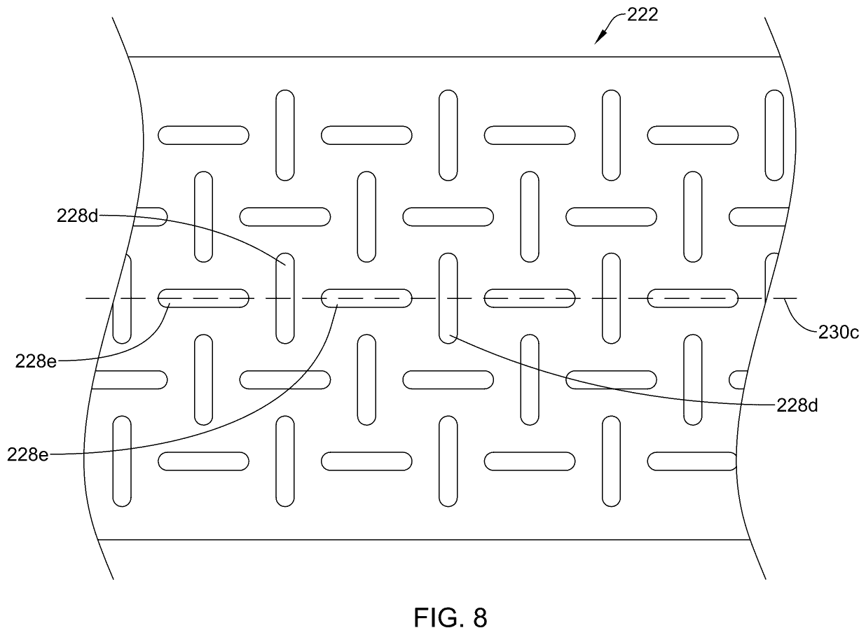

FIG. 8 illustrates another example interlock pattern including a plurality of individual slots 228d and a plurality of slots 228e formed along the guidewire shaft 222. It can be appreciated that the slots 228d and 228e may be similar in form and function to the slots 228a described herein. FIG. 8 illustrates that each individual slot 228d may be oriented generally perpendicular to the longitudinal axis 250 of the wall of the wall of the guidewire shaft 222. However, FIG. 8 further illustrates that each individual slot 228e may be oriented generally parallel to the longitudinal axis 250 of the wall of the guidewire shaft 222.

Further, the individual slots 228d and 228e forming the interlock pattern shown in FIG. 8 may be aligned in one or more rows extending generally parallel to the longitudinal axis 250 of the wall of the guidewire shaft 222. For example, FIG. 8 illustrates a plurality of individual slots 228d and 228e aligned along a first row 230c. Further, FIG. 8 illustrates that, in some examples, an individual slot 228d and individual slot 228e may alternate with one another along a given row. For example, FIG. 8 illustrates an alternating pattern of a slot 228d with a slot 228e along the row 230c. This is not intended to be limiting. Rather, any alternating pattern of slots 228d and slots 228e is contemplated.

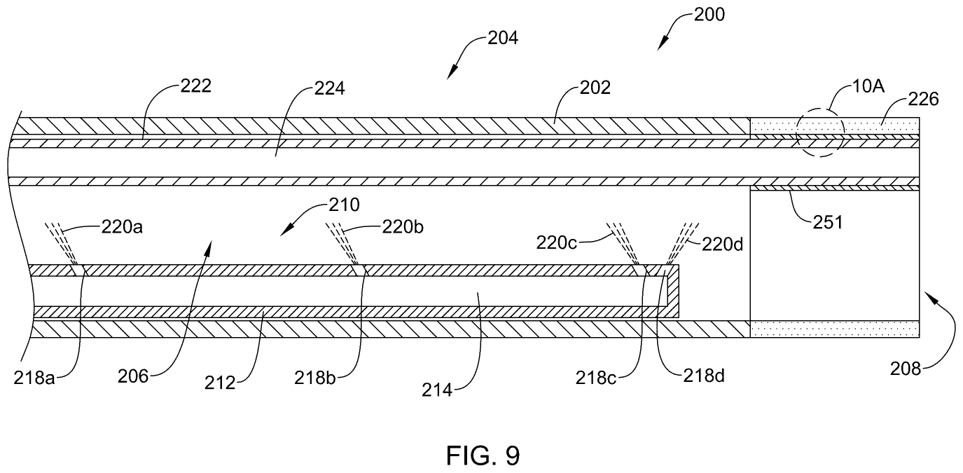

FIG. 9 is a cross-sectional view of a distal end region 204 of the thrombectomy catheter 200 shown in FIG. 3 prior to the bonding sleeve 251 and the tip member 226 being attached to the guidewire shaft 222. In other words, FIG. 9 illustrates an example manufacturing step in which both the bonding sleeve 251 and the tip member 226 have been positioned over top the guidewire shaft 222 prior to the bonding sleeve 251 and the tip member 226 being attached to the guidewire shaft 222.

FIG. 10A illustrates a detailed view of the thrombectomy catheter 200 shown in FIG. 9 prior to the bonding sleeve 251 and the tip member 226 being attached to the guidewire shaft 222. FIG. 10A illustrates a side of a plurality of individual slots 228a which form the interlock pattern (e.g., tip engagement pattern, slotted pattern, micro-slotted pattern, etched pattern, etc.) described herein with respect to FIG. 5. FIG. 10A further illustrates that each individual slot 228a forming the interlock pattern may extend from an outer surface of the guidewire shaft 222 into the wall of the guidewire shaft 222 a depth Y. In some examples, the depth Y of an individual slot 228a may be about 0.00009″ to about 0.002″, or about 0.000095″ to about 0.0015″, or about 0.0001″ to about 0.0012″, or about 0.0002″ to about 0.0008″, or about 0.0004″. FIG. 10A further illustrates that each individual slot 228a may be spaced from an adjacent slot 228a a distance Z. The spacing Z between individual slots 228a may be about 0.000095″ to about 0.003″, or about 0.0001″ to about 0.002″, or about 0.0005″ to about 0.0015″, or about 0.001″.

FIG. 10B illustrates another example embodiment of the detailed side view of the thrombectomy catheter 200 shown in FIG. 9 prior to the bonding sleeve 251 and the tip member 226 being attached to the guidewire shaft 222. For simplicity, it is noted that the bonding sleeve 251 shown in FIG. 10A has been omitted from FIG. 10B. It can be appreciated that the bonding sleeve 251 may be positioned between the tip member 226 and the guidewire shaft 222 similar to that shown in FIG. 10A. FIG. 10B illustrates a side of a plurality of individual slots 228a which form the interlock pattern (e.g., tip engagement pattern, slotted pattern, micro-slotted pattern, etched pattern, etc.) described herein with respect to FIG. 5. FIG. 10B further illustrates that each individual slot 228a forming the interlock pattern may extend radially inward at an offset angle relatively to an axis extending substantially perpendicular to the longitudinal axis of the guidewire shaft 222. In other words, each individual slot 228a forming the interlock pattern may extend radially inward at an offset angle whereby each individual slot 228a is angled toward the distal end of the tip member 226.

FIG. 10C illustrates another example embodiment of the detailed side view of the thrombectomy catheter 200 shown in FIG. 9 prior to the bonding sleeve 251 and the tip member 226 being attached to the guidewire shaft 222. For simplicity, it is noted that the bonding sleeve 251 shown in FIG. 10A has been omitted from FIG. 10C. It can be appreciated that the bonding sleeve 251 may be positioned between the tip member 226 and the guidewire shaft 222 similar to that shown in FIG. 10A. FIG. 10C illustrates a side of a plurality of individual slots 228a which form the interlock pattern (e.g., tip engagement pattern, slotted pattern, micro-slotted pattern, etched pattern, etc.) described herein with respect to FIG. 5. FIG. 10C further illustrates that each individual slot 228a forming the interlock pattern may extend radially inward at an offset angle relatively to an axis extending substantially perpendicular to the longitudinal axis of the guidewire shaft 222. In other words, each individual slot 228a forming the interlock pattern may extend radially inward at an offset angle whereby each individual slot 228a is angled away from the distal end of the tip member 226.

It can be appreciated that the interlock pattern discussed herein may also include individual slots 228a whereby some of the slots 228a may extend radially inward an offset angle which is angled toward the distal end of the tip member 226 (such as those shown in FIG. 10B) while also including some slots 228a which extend radially inward an offset angle which is angled away from the distal end of the tip member 226 (such as those shown in FIG. 10C). For example, the interlock pattern discussed herein may include a slot 228a which is angled toward the distal end of the tip member 226 positioned adjacent to a slot 228a which is angled away from the distal end of the tip member 226. Further, an alternating pattern of a slot 228a angled toward and a slot 228a angled away from the distal end of the tip member 226 may extend throughout the entire interlock pattern.

FIG. 11 illustrates a detailed side view of the thrombectomy catheter 200 shown in FIG. 10 after the bonding sleeve 251 has been attached to the guidewire shaft 222. FIG. 11 illustrates the bonding sleeve 251 having been heated (e.g., via a laser reflow process, a hot jaw/clamshell process, IR heat process, etc.) to a temperature in which the polymer forming the bonding sleeve reflows into the individual slots 228a forming the interlocking pattern described herein. It can be appreciated that after being reflowed and cooling, the polymer forming the bonding sleeve 251 may be mechanically engaged (e.g., mechanically locked) to the guidewire shaft 222, thereby providing an increased bond strength between the tip member 226 and the guidewire shaft 222.

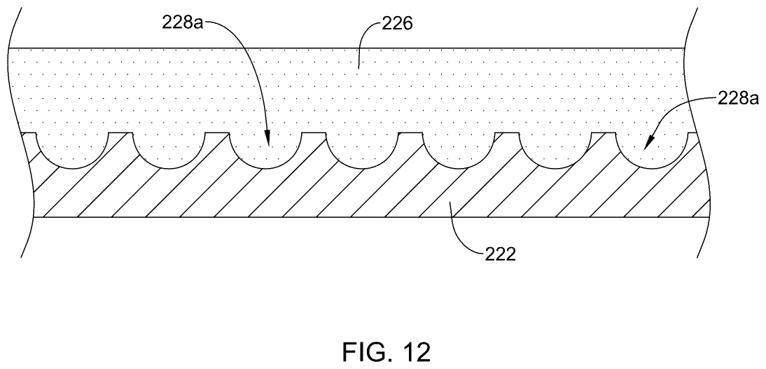

FIG. 12 illustrates a detailed view the thrombectomy catheter 200 shown in FIG. 5 after the tip member 226 has been attached to the bonding sleeve 251 and/or the guidewire shaft 222. FIG. 12 illustrates the tip member 226 having been heated (e.g., via a laser reflow process, a hot jaw/clamshell process, IR heat process, etc.) to a temperature in which the polymer forming the tip member 226 combines with the material of the bonding sleeve 251 and/or reflows into the individual slots 228a forming the interlocking pattern described herein. It can be appreciated that after being reflowed and cooling, the polymer forming the tip member 226 may be mechanically engaged (e.g., mechanically locked) to the bonding sleeve 251 and/or the guidewire shaft 222, thereby providing an increased bond strength between the tip member 226 and the guidewire shaft 222. This increased bond strength allows the guidewire shaft 222 to withstand the forces of the jetted motive fluid 220a-d leaving the jet orifices 218a-d. It can further be appreciated that after the tip member 226 has been reflowed, the distal end of the thrombectomy catheter 200 may be skive cut to form the angled (e.g., tapered) tip shown in FIG. 3.

The materials that can be used for the various components of the catheter 200 may include those commonly associated with medical devices. However, this is not intended to limit the devices and methods described herein, as the discussion may be applied to other components, devices, or systems disclosed herein.

The components of the catheter 200 (and/or other systems disclosed herein) may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the components of the catheter 200 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the components of the catheter 200 (and/or other systems disclosed herein) in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the components of the system 10 (and/or other systems disclosed herein) to achieve the same result.

In some embodiments, a degree of Magnetic Resonance Imaging (MRI) compatibility is imparted into the catheter 200 (and/or other systems disclosed herein). For example, components of the catheter 200 (and/or other systems disclosed herein), may be made of a material that does not substantially distort the image and create substantial artifacts (e.g., gaps in the image). Certain ferromagnetic materials, for example, may not be suitable because they may create artifacts in an MRI image. The components of the catheter 200 (and/or other systems disclosed herein) or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An aspiration catheter, comprising:
a catheter shaft having a proximal end region, a distal end region and a lumen extending therein;
a tip member having a length and a lumen extending therein, wherein the tip member is coupled to the distal end region of the catheter shaft; and
a guidewire shaft having a proximal end region and a distal end region;
wherein the guidewire shaft extends within at least a portion of the lumen of the catheter shaft and the lumen of the tip member;
wherein the distal end region of the guidewire shaft includes an interlock region, and wherein a portion of the tip member is engaged with at least a portion of the interlock region.

2. The aspiration catheter of claim 1, wherein the interlock region includes a plurality of discrete slots.

3. The aspiration catheter of claim 2, wherein each of the discrete slots includes an oval shape defined along an outer surface of the guidewire shaft.

4. The aspiration catheter of claim 2, wherein each of the discrete slots extends radially inward from an outer surface of the guidewire shaft.

5. The aspiration catheter of claim 2, wherein the plurality of discrete slots are aligned to form one or more rows of slots extending along a longitudinal axis of a wall of the guidewire shaft.

6. The aspiration catheter of claim 5, wherein the plurality of discrete slots are aligned to form a first column of slots positioned adjacent a second column slots, and wherein the first column of slots is circumferentially offset from the second column of slots.

7. The aspiration catheter of claim 2, wherein one or more of the discrete slots is oriented at an oblique angle relative to a longitudinal axis of a wall of the guidewire shaft.

8. The aspiration catheter of claim 4, wherein a portion of the tip member is configured to extend into each of the discrete slots of the interlock region.

9. The aspiration catheter of claim 8, wherein extension of the tip member into each of the discrete slots forms a mechanical bond between the tip member and the guidewire shaft.

10. The aspiration catheter of claim 1, wherein the interlock region includes a length extending proximally from the distal end of the guidewire shaft, and wherein the length of the interlock region is substantially equal to the length of the tip member.

11. The aspiration catheter of claim 1, wherein the interlock region includes a length extending proximally from the distal end of the guidewire shaft, and wherein the length of the interlock region is less than the length of the tip member.

12. The aspiration catheter of claim 1, wherein a portion of the proximal end region of the guidewire shaft is attached to a manifold, and wherein at least a portion of the guidewire shaft extending between the manifold and the interlock region remains unattached to the catheter shaft.

13. A thrombectomy system, comprising:
a processor coupled to a pump and a thrombectomy catheter, wherein the thrombectomy catheter includes:
an outer shaft having a proximal end region attached to a manifold, a distal end region and a lumen extending therein;
a tip member having a length and a lumen extending therein, wherein the tip member is coupled to the distal end region of the outer shaft; and
a guidewire shaft having a proximal end region and a distal end region;
wherein the guidewire shaft extends within at least a portion of the lumen of the outer shaft and the lumen of the tip member;
wherein the distal end region of the guidewire shaft includes an interlock region, and wherein a portion of the tip member is engaged with at least a portion of the interlock region of the guidewire shaft.

14. The thrombectomy system of claim 13, wherein the interlock region includes a plurality of discrete slots.

15. The thrombectomy system of claim 14, wherein each of the discrete slots includes an oval shape defined along an outer surface of the guidewire shaft.

16. The thrombectomy system of claim 15, wherein the plurality of discrete slots are aligned to form a first column of slots positioned adjacent a second column slots, and wherein the first column of slots is circumferentially offset from the second column of slots.

17. The thrombectomy system of claim 14, wherein each of the discrete slots extends radially inward from an outer surface of the guidewire shaft.

18. The thrombectomy system of claim 17, wherein extension of the tip member into each of the discrete slots forms a mechanical bond between the tip member and the guidewire shaft.

19. The thrombectomy system of claim 13, wherein at least a portion of the guidewire shaft extending between the manifold and the interlock region remains unattached to the outer shaft.

20. A method of manufacturing a thrombectomy catheter, the method comprising:
positioning an outer shaft adjacent a tip member, whereby a lumen of the outer shaft is in fluid communication with a lumen of the tip member;
positioning a guidewire shaft within the lumen of the outer shaft and the lumen of the tip member, wherein the guidewire shaft includes an interlock region having a plurality of discrete slots extending radially inward from an outer surface of the guidewire shaft;
aligning the guidewire shaft relative to the tip member such that a portion of the interlock region is positioned adjacent to the tip member; and
reflowing the tip member such that a portion of the tip member extends into each of the discrete slots of the interlock region.
